(19) United States
(12) Patent Application Publication
(10) Pub. No.: US 2007/0054401 A1
(43) Pub. Date: Mar. 8, 2007

(54) Composition for intracellular transport of biological particles or macromolecules

(75) Inventors: Alain Prochiantz, Paris (FR); Edmond Dupont, Paris (FR); Alain Joliot, Paris (FR); Alain Trembleau, Yerres (FR); Michel Volovitch, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: Centre National De La Recherche Scientifique, Paris (FR); Ecole Normale Superieure, Paris (FR)

(21) Appl. No.: 10/541,594

(22) PCT Filed: Dec. 31, 2003
(86) PCT No.: PCT/FR03/03951

§ 371 Date: Jan. 5, 2006

(30) Foreign Application Priority Data
Jan. 7, 2003 (FR) 03/00093

(51) Int. Cl. C12N 15/74

(52) U.S. Cl. 435/472; 977/803

(57) ABSTRACT

The invention relates to a composition comprising a macromolecule or particle having one or several hydrophobic domains on the surface thereof whereby at least one transducer peptide is adsorbed thereon. Said composition can be used to introduce the macromolecule or particle into living cells.

The present invention relates to novel means for the intracellular transfer of macromolecules or particles of interest.

The import of macromolecules, and in particular of polynucleotides or proteins, into living animal cells constitutes a basic approach both for basic research and in the context of various applications, for example in gene therapy.

One of the major difficulties of this approach results from the need to transport these macromolecules across the cell membrane.

This problem has been the subject of numerous research studies which have resulted in the development of various methods of intracellular transfer and of various types of vector.

Thus, the introduction of polynucleotides into cells is currently based mainly on techniques of transfection (calcium phosphate, electroporation), of lipofection (liposomes, charged lipids) or of viral infection (lentivirus, adenovirus, herpesvirus and the like) or on the use of nanoparticles.

More recently, it has been proposed to use transducing peptides. This term denotes peptides comprising, or consisting of, a sequence called “transduction domain” which confers on them the capacity to penetrate inside a living cell independently of the presence of specific transporters or receptors.

Review articles relating to transducing peptides have been recently published by LIDGREN et al., TiPS, 21, 99-102, (2000); SCHWARZE and DOWDY TiPS, 21, 45-48, (2000); SCHWARZE et al. Trends Cell. Biol., 10, 290-295, (2000); PROCHIANTZ Current Opinion in Cell Biology, 12, 400-406, (2000); Cell-Penetrating Peptides. Processes and applications. Ed. Ulo Langel. CRC Press (2002).

As examples of transducing peptides, there may be mentioned in particular:
penetratins, which are peptides derived from the third helix of a homeodomain; peptides of the penetratin family are described for example in the publications by JOLIOT et al., Proc. Natl. Acad. Sci. USA, 88, 1864-1868, (1991); DEROSSI et al., J. Biol. Chem., 269, 14, 10444-10450, (1994); BRUGIDOU et al. Biophys. Biochem. Res. Com., 214, 685-693, (1995), and in U.S. Pat. No. 5,888,762, U.S. Pat. No. 6,080,724, or PCT application WO 00/01417;
peptides derived from the HIV1 Tat protein, and in particular from fragment 48-60 of said protein; such peptides are described for example by FAWELL et al. Proc. Natl. Acad. Sci. USA., 91, 664-668, (1994) or by VIVES et al. J. Biol. Chem., 272, 16010-16017, (1997);
peptides derived from the HSV VP22 protein; such peptides are described for example by ELLIOTT and O'HARE Cell, 88, 223-233, (1997);
peptides derived from a signal sequence conjugated with a nuclear localization sequence; such peptides are described for example by LIN et al. J. Biol. Chem., 270, 14255-14258, (1995); J. Biol. Chem., 271, 5305-5308, (1996), LIU et al. Proc. Natl. Acad. Sci. USA, 93, 11819-11824, (1996), MORRIS et al. Nucleic Acids Res., 25, 2730-2736, (1997), CHALOIN et al. Biochemistry, 36, 11179-11187, (1997); Biochem. Biophys. Res. Commun., 243, 601-608, (1998), ZHANG et al. Proc. Natl. Acad. Sci. USA, 95, 9184-9189, (1998);
transportans which are derived from a fusion between a portion of a neuropeptide, galanin, and a wasp venom peptide; POOGA et al., FASEB J., 12, 67-77, (1998); Ann. New York Acad. Sci., 863, 450-453, (1998).

The transducing peptides may import into living cells, in particular animal cells, molecules or molecular complexes of a diverse nature (nucleic acids, proteins, peptides/nucleic acids, nucleotide analogs, liposomes).

These molecules or molecular complexes are usually designated under the general term of “cargos”.

It has been reported that some transducing peptides can import cargos of a large size.

Thus, LEWIN et al. (Nat. Biotech, 18, 410-414, 2000) conjugated a derivative of the transducent peptide TAT 48-60 with nanoparticles consisting of an iron oxide core coated with a dextran envelope, and observed that the nanoparticles thus modified (having a diameter of 45 nm) were imported into living cells.

EGUCHI et al. (J. Biol. Chem., 276, 28, 26204-26210, 2001) constructed recombinant λ phages expressing, at their surface, a chimeric protein comprising the transducing peptide TAT fused with the N-terminal end of the D protein of the phage, and containing a marker gene. They observed, after incubating these phages with COS-1 cells in culture, an intracellular expression of the marker gene in a proportion of these cells which could be up to 30%.

It is however generally considered that one of the major limitations of the transducing peptides mentioned above, such as penetratins or TAT peptides, lies in the need to couple the transducing peptide and the cargo by (a) covalent bond(s).

With the aim of dispensing with this limitation, peptides designed to bind through ionic or hydrophobic interactions either to nucleic acids or to proteins have been constructed. One of these peptides, called MPG, is intended for the intracellular transport of nucleic acids (MORRIS et al., Nucl. Acids Res., 2730-2736, 1997; Nucl. Acids Res., 3510-3517, 1999); it comprises two distinct regions, separated by a linking peptide: an N-terminal hydrophobic region derived from the signal sequence rich in glycine of the gp41 protein of HIV1, which allows fusion with the cell membrane, and a hydrophilic region derived from the nuclear localization sequence of the SV40 T antigen, which allows interaction of the peptide with the nucleic acid, and its nuclear targeting.

The other, called Pep-1 (MORRIS et al., Nature Biotech, 19, 1173-1176, 2001) is intended for the transport of proteins. It differs from MPG by the nature of the N-terminal hydrophobic region, which consists of a sequence rich in tryptophan, intended to allow targeting to the cell membrane and the formation of hydrophobic interactions with the proteins.

These two types of peptide are also described in PCT application WO 02/10201, which proposes, in a general manner, to use, in order to import proteins into living cells, peptides of 16 to 30 amino acids comprising two distinct successive domains: a hydrophobic domain containing 3 to 5 tryptophan residues including at least one Trp-Trp pair, alternating with glutamic acid and threonine residues; a hydrophilic domain containing 4 or 5 consecutive basic residues (lysine or arginine), these two domains being possibly separated by a spacer domain containing a proline residue or a glutamine residue. An effective import was however observed only in the case of peptides additionally bearing a cysteamine group.

Moreover, in the context of the work on the properties of transducing peptides of the penetratin family, the inventors evaluated the capacity of these peptides to import cargos of a large size. With this aim in mind, they tested one of these peptides using a λ phage as cargo. They then observed not only that this peptide was capable of importing the phage into an animal cell, but also that, contrary to what was assumed or wanted now, the import could take place without the need to couple the peptide and the phage by a covalent bond. In addition, the inventors observed that the efficacy of this import was much higher than that observed by EGUCHI et al. with the transducing peptide TAT coupled by a peptide bond to the N-terminal end of the λ phage D protein.

To explain these results, which are surprising in the light of the structural difference between the penetratins and the peptides of the PCT application WO 02/10201, the inventors propose the following hypothesis: penetratins have a transduction domain capable of adopting an amphiphilic secondary structure (in the form of an α helix or in the form of a β sheet) possessing a surface having hydrophobic residues, and a charged surface comprising a tryptophan residue flanked by 2 basic residues bringing about the interaction with the membranes and the formation of a reverse micelle allowing internalization of the peptide in the cell. For example, in the case of the penetratin-type pANTP, the Ile, Trp and Phe residues at positions 3, 14, and 7 of the peptide sequence form a hydrophobic triplet in the α helix; this hydrophobic triplet is distant from the charged zone consisting of the Lys residue (position 13 of the peptide sequence) and Arg residue (position 10 of the peptide sequence), which, in the α helix, flank the Trp residue at position 6 of the peptide sequence (DEROSSI et al. J. Biol. Chem, 271, p. 18188-18193, 1996).

It is assumed that the hydrophobic surface of the transduction domain allows the formation of interactions of sufficient strength to bring about a stable attachment of the transducing peptide to the cargo. The interaction with the membrane is thought to occur through the charged surface of the transduction domain; the Trp flanked by two charged amino acids can insert itself into the membrane, (this insertion was observed by fluorescence studies of tryptophan), destabilizing it and allowing the passage of the vector and its cargo.

The subject of the present invention is a method for preparing a composition which makes it possible to introduce into a living cell, and in particular a eukaryotic cell, and especially an animal cell, a cargo consisting of a macromolecule or a molecular assembly (for example a particle), having a size of less than or equal to about 1 μm along its largest dimension, said cargo having one or more hydrophobic domains at its surface, said method is characterized in that it comprises the adsorption, onto said hydrophobic domain(s), of at least one transducing peptide, with the exception of the peptides described in PCT application WO 02/10201.

According to a preferred embodiment of the present invention, said cargo is a protein or a particle having a surface of a proteic nature.

It is also possible to use, as cargo, liposomes, nanoparticles, glycolipids or any other natural or artificial macromolecular combination.

According to another preferred embodiment of the present invention, said cargo generally has a size of less than or equal to 500 nm along its largest dimension.

This includes for example viral or pseudoviral particles, in particular phage particles.

According to yet another preferred embodiment of the present invention, said transducing peptide is a peptide of the penetratin family.

The term “peptide of the penetratin family” is defined here as any peptide comprising a transduction domain capable of adopting an amphiphilic secondary structure (in the form of an a helix or in the form of a β sheet) which has a surface comprising hydrophobic residues allowing interaction with the cargo, and a surface allowing interaction with the membranes, comprising a tryptophan residue flanked by basic residues.

This includes in particular the penetratins described in PCT application WO 00/01417, and more particularly those comprising a transduction domain defined by one of the formulae below:
X₁-X₂-X₃-X₄-X₅-X₆-X₇-X₈-X₉-X₁₀-X₁₁-X₁₂-X₁₃-X₁₄-X₁₅-X₁₆ (I)
X₁₆-X₁₅-X₁₄-X₁₃-X₁₂-X₁₁-X₁₀-X₉-X₈-X₇-X₆-X₅-X₄-X₃-X₂-X₁ (Ia)
in which X₆ represents a tryptophan residue, X₁, X₂, X₄, X₉, X₁₅, X₁₆, are nonhydrophobic amino acids, and X₃, X₇, and X₁₄ are hydrophobic amino acids.

Particularly preferred transduction domains for carrying out the present invention are those in which X₁₀ and X₁₃ are basic amino acids.

It is also possible to use penetratin derivatives, for example certain truncated or substituted penetratins described in PCT application WO 00/01417, or PCT application WO 00/29427.

It is also possible to use a transducing peptide comprising, in addition to the transduction domain, one or more other functional domains; by way of example, there may be mentioned peptides comprising a transduction domain and a nuclear export sequence which are described in PCT application WO 02/39947.

The adsorption of the transducing peptide takes place in a simple manner, by incubating said transducing peptide with the cargo for at least 15 minutes, preferably for 30 to 60 minutes.

The incubation can take place ex vivo or in vivo, in a very broad temperature range generally between 15 and 40° C. The procedure will be preferably carried out at room temperature, that is to say in the region of 20 to 25° C., or at physiological temperatures (in the region of 37° C.), in a medium at neutral pH; this may be for example a cell culture medium, or an NaCl solution (9 g/l).

The transducing peptide/cargo molar ratio in the incubation medium depends in particular on the size of the cargo; for example, in the case of a bacteriophage, it is possible to use a molar ratio corresponding to 1000 to 500 000 molecules of peptide per bacteriophage.

The subject of the present invention is also a composition comprising a cargo at the surface of which a transducing peptide capable of being obtained by the method in accordance with the invention is adsorbed.

The compositions in accordance with the invention may be used immediately after their preparation; where appropriate, they may also be stored for at least three days in the incubation medium, at temperatures of between 4° C. and 37° C. approximately.

The subject of the present invention is also the use of compositions in accordance with the invention for introducing a cargo, as defined above, into a living cell.

In particular, the subject of the present invention is thus a method for introducing a cargo into a living cell, characterized in that it comprises bringing said cell into contact with a composition in accordance with the invention comprising said cargo.

The method in accordance with the invention may be performed on cells in culture, by adding to the culture a composition in accordance with the invention, and incubating for 1 to 14 hours, preferably for 2 to 6 hours.

Preferably, the composition in accordance with the invention is used in an amount of 10 000 to 20 000 cargo/transducing peptide complexes per cell.

The method in accordance with the invention may also be performed in vivo, for example by injecting a composition in accordance with the invention into an animal.

The subject of the present invention is also the use of a composition in accordance with the invention for producing a medicament, and in particular as a vector for an active ingredient consisting of the cargo or contained therein.

The present invention has the advantage of allowing the introduction into living cells of any hydrophobic cargo or any cargo whose surface has at least one hydrophobic domain, without the need to perform preliminary coupling by a covalent bond between the cargo and the transducing peptide. The present invention has a very special advantage for introducing, into living cells, viral or pseudoviral particles, in particular bacteriophages, containing polynucleotides of interest which it is desired to express in said cells.

These particles may thus be used for example as vectors for gene therapy in vivo or ex vivo. It may also be possible to prepare compositions in accordance with the invention from phage libraries containing polynucleotides encoding various polypeptides capable of modifying the behavior of certain cells (migration, proliferation, differentiation and the like), and to use these compositions to cause these phage libraries to enter into tissues, in culture or in vivo, and to identify sequences regulating these behaviors.

The present invention will be understood more clearly with the aid of the additional description which follows, which refers to nonlimiting examples illustrating the use of the present invention to introduce phages into living cells.

### EXAMPLE 1

### Adsorption of a Transducing Peptide onto Lamda Bacteriophages

Preparation of the phages:

The gene for the autofluorescent protein EGFP (CLONTECH) or that for the homeoprotein En2 (Engrailed2) from chicken (LOGAN et al., 1992, Dev Genetics 13: 345-358) were placed under the control of the CMV promoter and upstream of the SV40 polyadenylation sequence, in a plasmid derived from pBK-CMV (STRATAGENE) possessing a unique EcoRI site upstream of the CMV promoter, and a unique SalI site downstream of the polyadenylation signal.

The functionality of these constructs was verified by electroporation and transient expression in COS cells, and detection of the autofluorescence of GFP or immunocytochemical detection of the Engrailed 2 protein with the aid of a polyclonal antibody directed against this protein (gift from Dr S. SAULE, UMR 146, Institut Curie, Orsay)).

The fragment flanked by the two unique sites (EcoRI and SalI) was then transferred into the genome of the Lambda-ZAP phage (STRATAGENE), between the EcoRI and XhoI sites, and the recombinant DNA was encapsidated in vitro with the aid of the GIGAPACK PLUS reagents (STRATAGENE). The resulting phages (called Lambda-ZAP-GFP and Lambda-ZAP-En2 respectively) allowed the infection of competent bacteria (XL1Blue-MRF′ strain, STRATAGENE), and their titer was then determined and they were stored after a first round of amplification. To check the quality of the recombinants obtained, the phagemids within the genomes of the recombinant Lambda phages were automatically excised by co-infection of XL1Blue-MRF′ bacteria with a helper phage (ExAssist, STRATAGENE). After culturing in liquid medium, the bacteria which were still alive and the Lambda virions were destroyed by heating, and the recombinant filamentous phages are recovered. The plasmid forms of these recombinant phagemids are recovered after infection of nonpermissive bacteria for the replication of the filamentous phage (SOLR strain, STRATAGENE), and the functional integrity of these excised plasmids is verified by electroporation in COS cells. After this verification, the recombinant lambda phages are amplified in order to reach a titer of at least 10¹¹ particles per ml, and then concentrated using PEG, dialyzed against PBS supplemented with Ca⁺⁺ and Mg⁺⁺, and stored at 4° C.

Adsorption of the Transducing Peptide:

The transducing peptide used is a penetratin having the sequence:

| | |
|---|---|
| RQIKIWFQNRRMKWKK | (SEQ ID NO:1) |

corresponding to helix 3 of the pAntp peptide (homeodomain of the Drosophila Antennapedia protein).

The biotinylated penetratin is mixed with the recombinant phages in an amount of 10 μg of peptide per 10⁹ phage particles, in 50 to 100 μl of appropriate medium (DMEM/F12 medium (1:1) or PBS-Dulbecco medium). The mixture is incubated at room temperature for 30 min.

### EXAMPLE 2

### Introduction of Phages into Cells in Culture

The cells used are dog kidney epithelial cells (MDCK).

The phages used are labeled with the fluorochrome Cy3 (AMERSHAM) by covalent bonding of the fluorochrome to the capsid proteins, according to the manufacturer's instructions. They are then incubated in the presence of penetratin, as described in Example 1 above. As negative control, phages labeled with the fluorochrome Cy3 are used, which are incubated under the same conditions in the absence of penetratin.

The phage/penetratin preparation, or the control preparation is added to the culture or cell suspension medium (depending on whether the treated cells have already been inoculated or whether they have just been dissociated) in an amount of 10 000 phages/cell, and left in contact with the cells for 4 hours.

The cells are then washed and resuspended in fresh medium, and then fixed in 4% paraformaldehyde in PBS for 10 min at room temperature, rinsed in PBS and mounted in mounting medium for fluorescent specimens DAKO containing 1 μg/ml of DAPI (4′-6-diamidino-2-phenylindole). They are then observed under a Leica TCS type epifluorescence confocal microscope. The images are analyzed and processed with the aid of the Adobe Photoshop software.

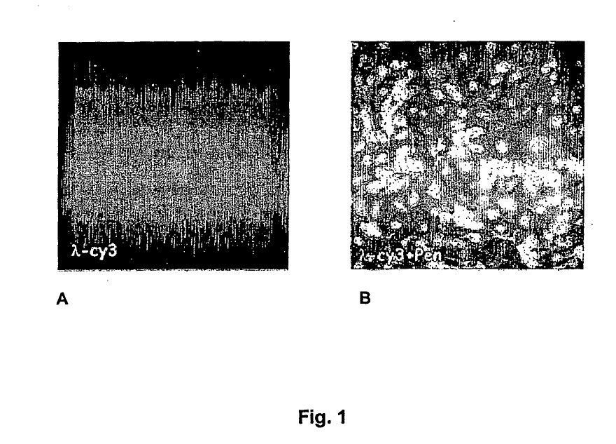

The results are illustrated in FIG. 1:

FIG. 1A: control preparation with phage without penetratin.

FIG. 1B: phage/penetratin preparation.

A high intracellular fluorescence is observed in the cells which received the phage/penetratin preparation. On the other hand, in the case of the cells which received only the phage preparation, no fluorescence is observed.

### EXAMPLE 3

### Introduction of Lambda Phages in vivo into Mouse Brain Cells

Various phage/penetratin preparations (recombinant phages expressing GFP; recombinant phages exprssing En2; phages labeled with the fluorochrome Cy3) are administered to adult mice by infusion into the lateral ventricle of the brain.

On D-1 before the infusion, the phages (solution at 6.5×10⁸ pfu/μl) are dialyzed against 0.9% NaCl containing 10 mM of MgCl₂ (for the stability of the phage) at 4° C. overnight. On the day of the infusion, the phage/penetratin mixture is prepared: 70 μl of the solution of phages dialyzed against 0.9% NaCl (that is 6.5×10¹⁰ pfu)+3 μl of 9% NaCl+27 μl of the penetratin stock solution (that is 162 μg), that is about 5×10⁵ molecules of penetratin per phage particle. 100 μl of mixture are loaded into an osmotic micropump (ALZET 1003D) collected by a catheter to a cannula which will be implanted into the lateral ventricle. The whole micropump is immersed in 0.9% NaCl at 37° C. for 4 hours in order to initiate its rate of flow.

The pumps are placed in a subcutaneous pouch at the level of the scapular region of the animal, and the canula is implanted into the lateral ventricle of the brain according to the following stereotaxic coordinates: lateral 0.8 mm, anteroposterior 0 mm, dorsoventral 2 mm relative to the Bregma of the skull taken as origin of the coordinates.

The infusion is performed for three days at a flow rate of 1 μl/hour. The infused animals are then humanely killed by anesthesia followed by intracardiac infusion of 4% paraformaldehyde in PBS; the brains are removed and post-fixed overnight at 4° C. in this fixative. The next day, they are cut using a vibratome into frontal sections 50 μm thick.

The sections are either observed immediately after mounting in mounting medium (DAKO+DAPI) in the case of direct fluorescence (GFP or CY3), or used for the immunodetection of the heterologous protein expressed by the phage (in the case of the phage expressing GFP or En2). The penetratin is detected by a streptavidin coupled to the fluorochrome Cy3 (IMMUNOTECH).

For the immunodetection, and/or the detection of the penetratin, the sections are preincubated for about one hour in PBS buffer containing 5% FCS and 0.25% Triton X-100 (PBST) at room temperature. The antibodies are diluted in the same buffer, at 1/5000 for the anti-En2 polyclonal antibody, and at 1/500 for the anti-GFP polyclonal antibody (SANTA-CRUZ), and incubated with the sections overnight at 4° C. The sections are then rinsed 3×15 min in PBS buffer; an FITC-coupled fluorescent secondary antibody to rabbit immunoglobulins (JACKSON) is then added after 1/500 dilution in PBST.

For the detection of penetratin, the fluorescent streptavidin is diluted 1/500 in PBST.

After incubating for 1 hour at room temperature, and three 15 min rinses in PBS, the sections are mounted in DAKO+DAPI medium, and observed by epifluorescence confocal microscopy.

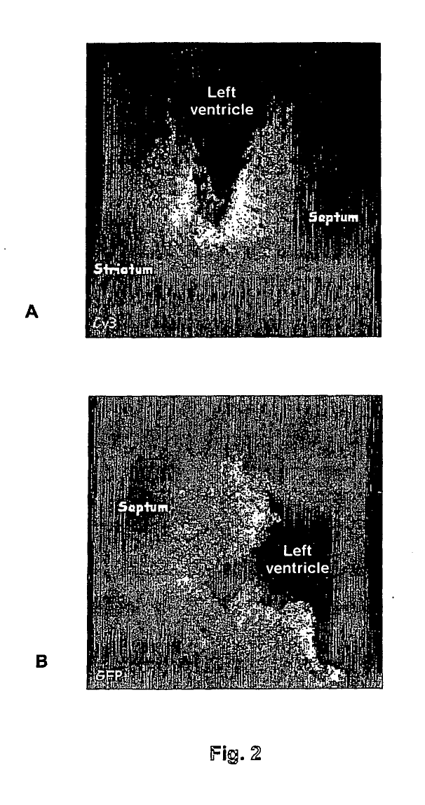

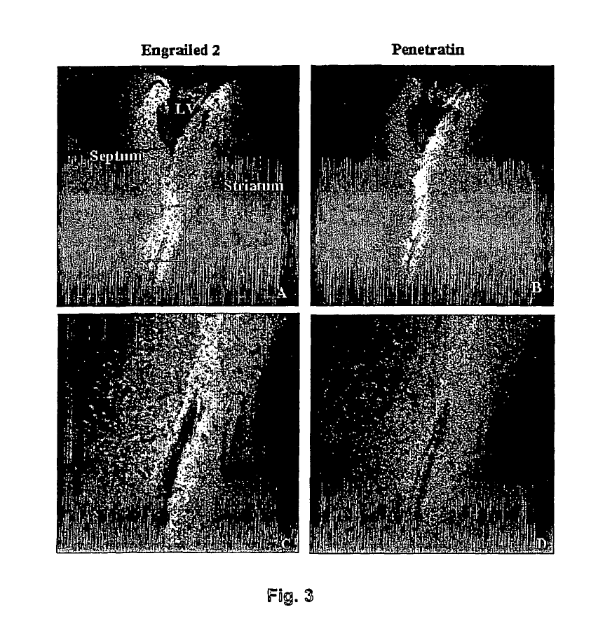

The results are illustrated in FIGS. 2 and 3:

FIGS. 2A and 2B represent labelings on frontal sections 50 μm thick.

FIG. 2A: Detection of Cy3 phage in cerebral parenchyma of an adult mouse after infusion of the penetratin/phage mixture into the lateral ventricle.

FIG. 2B: Detection of a GFP fluorescence in the cerebral parenchyma of an adult mouse after infusion of the penetratin/GFP phage mixture into the lateral ventricle.

FIG. 3: Colocalization of the Engrailed 2 protein and of penetratin in the cerebral parenchyma of an adult mouse after infusion of the penetratin/phage encoding En 2 mixture.

FIG. 3A: Immunodetection of the Engrailed 2 protein encoded by the phage.

FIG. 3B: detection on the same section of penetratin with the aid of fluorescent streptavidin.

FIGS. 3D and 3E are magnifications of FIGS. 3A and 3B respectively.

1. A method for preparing a composition, comprising:
contacting at least one transducing peptide, having at least one hydrophobic domain with a
a cargo consisting of a macromolecule or a molecular assembly having a size of less than or equal to about 1 μm along its largest dimension and having one or more hydrophobic domains at its surface, under conditions suitable for adsorption of the transducing peptide to at least one surface hydrophobic domain of the cargo,
said method is characterized in that it comprises the adsorption onto said hydrophobic domain(s) of at least one transducing peptide,
with the proviso that said transducing peptide is not 16-30 amino acid residues in length comprising a hydrophobic domain containing 3 to 5 tryptophan residues and at least one Trp-Trp pair, alternating with glutamic acid and threonine residues, and a hydrophilic domain containing 4 or 5 consecutive basic residues.

2. The method of claim 1, wherein the cargo is a protein or a particle possessing a surface of a proteic nature.

3. The method of claim 1, wherein the cargo is a viral or pseudoviral particle.

4. The method of claim 1, wherein the cargo is a bacteriophage.

5. The method of claim 1, wherein the transducing peptide is a peptide of the penetratin family.

6. The method of claim 1, wherein the adsorption of the transducing peptide is performed by incubating for at least 15 minutes said transducing peptide with the cargo.

7. A composition comprising:
a cargo consisting of a macromolecule or a molecular assembly having a size of less than or equal to about 1 μm along its largest dimension and having one or more hydrophobic domains at its surface, and
a transducing peptide attached to at least one surface hydrophobic domain of said cargo;
with the proviso that said transducing peptide is not 16-30 amino acid residues in length comprising a hydrophobic domain containing 3 to 5 tryptophan residues and at least one Trp-Trp pair, alternating with glutamic acid and threonine residues, and a hydrophilic domain containing 4 or 5 consecutive basic residues.

8. The composition of claim 7, wherein said transducing peptide comprises a transduction domain of SEQ ID NO: 2 or 3.

9. A pharmaceutical composition comprising the composition of claim 7.

10. A method for introducing a cargo into a living cell in culture comprising:
contacting said living cell with the composition of claim 7.

11. The method of claim 10, wherein said contacting occurs in vivo.

12. The method of claim 10, wherein said contacting occurs in vitro.

13. The method of claim 10, wherein said living cell is a eukaryotic cell.

14. The method of claim 10, wherein said cargo comprises a nucleic acid or nucleic acid analog.

15. The method of claim 10, wherein said cargo comprises a peptide or a protein.


SEQUENCE LISTING (3 sequences)

SEQ ID NO: 1 - protein, 16 aa, Drosophila melanogaster
  MISC_FEATURE (1)..(16): Helix 3 of the pAntp homeodomain
RQIKIWFQNRRMKWKK

SEQ ID NO: 2 - protein, 16 aa, Artificial
  Penetrin transduction domain motif.  Xaa′s at positions 1, 2, 4, 9, 15 and 16 are nonhydrophobic amino acids. Xaa′s at positions 3, 7 and 14 are hydrophobic amino acids.  Xaa′s at positions 5, 8, and 10-13 are any amino acid.
  misc_feature (1)..(5): Xaa can be any naturally occurring amino acid
  misc_feature (7)..(16): Xaa can be any naturally occurring amino acid
XXXXXWXXXXXXXXXX

SEQ ID NO: 3 - protein, 16 aa, Artificial
  Penetrin transduction domain motif.  Xaa′s at positions 1, 2, 8, 13, 15 and 16 are nonhydrophobic amino acids. Xaa′s at positions 3, 10 and 14 are hydrophobic amino acids. Xaa′s at positions 4-7, 9, and 12 are any amino acid.
  misc_feature (1)..(10): Xaa can be any naturally occurring amino acid
  misc_feature (12)..(16): Xaa can be any naturally occurring amino acid
XXXXXXXXXXWXXXXX